(19) United States
(12) Patent Application Publication
(10) Pub. No.: US 2018/0361097 A1
(43) Pub. Date: Dec. 20, 2018

(54) INTERFACE DEVICE HAVING USER SELECTED INDICIA FORMED THEREIN

(71) Applicant: KONINKLIJKE PHILIPS N.V., EINDHOVEN (NL)

(72) Inventor: Peter Chi Fai HO, PITTSBURGH, PA (US)

(21) Appl. No.: 16/062,208

(22) PCT Filed: Dec. 20, 2016
(86) PCT No.: PCT/EP2016/081885

§ 371 Date: Jun. 14, 2018

Related U.S. Application Data
(60) Provisional application No. 62/270,748, filed on Dec. 22, 2015.

(51) Int. Cl. A61M 16/06

(52) U.S. Cl. CPC A61M 16/0622; A61M 2016/0661; A61M 2205/60; A61M 2207/10; A61M 2205/583

(57) ABSTRACT

A customized member for use in an interface device for delivering a flow of breathing gas to the airway of a patient. The member includes at least one dimensional characteristic selected to fit the member to the patient and an indicia, which was selected by the patient or a representative thereof, formed permanently therein.

### CROSS-REFERENCE TO RELATED APPLICATIONS

This patent application claims the priority benefit under 35 U.S.C. § 119(e) of U.S. Provisional Application No. 62/270,748 filed on Dec. 22, 2015, the contents of which are herein incorporated by reference.

### BACKGROUND OF THE INVENTION

### 1. Field of the Invention

The present invention pertains to a customized member for use in an interface device for delivering a flow of breathing gas to the airway of a patient. The present invention also pertains to an interface device for use in delivering a flow of breathing gas to the airway of a particular patient having a customized member. The present invention further pertains to a method of producing a customized member for use in an interface device.

### 2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is OSA. Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve the placement of a patient interface device including a mask component having a soft, flexible cushion on the face of a patient. The mask component may be, without limitation, a nasal mask that covers the patient's nose, a nasal cushion having nasal prongs that are received within the patient's nares, a nasal/oral mask that covers the patient's nose and mouth, or a full face mask that covers the patient's face. Such patient interface devices may also employ other patient contacting components, such as forehead supports, cheek pads and chin pads. The patient interface device is connected to a gas delivery tube or conduit and interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

Because such masks are typically worn for an extended period of time, a variety of concerns must be taken into consideration. For example, in providing CPAP to treat OSA, the patient normally wears the patient interface device all night long while he or she sleeps. One concern in such a situation is that the patient interface device is as comfortable as possible; otherwise the patient may avoid wearing the interface device, defeating the purpose of the prescribed pressure support therapy. It is also important that the overall size of the interface device be minimized, so as to not be overly cumbersome on the face of the patient.

Historically, interface devices are produced in several sizes in order to accommodate the wide variety of facial sizes and structures present in the population. As the number of mask sizes provided increases, so does the costs associated with manufacturing, stocking, etc. Recent advancements in manufacturing processes have led to the ability to produce interface devices that can be customized in-whole or in-part to fit a particular patient. While such advancements have generally improved the fit and thus the comfort of such devices, patient compliance in wearing such devices is still a problem as patients tend to find such devices too clinical, impersonal, and lacking emotional appeal to the personal level to remind the patient that their mask is something prescribed especially for them to treat their conditions. Also, there is not enough market differentiation to distinguish a highly customized mask for a particular individual from mass-produced commercial masks.

### SUMMARY OF THE INVENTION

Accordingly, it is an object of the present invention to provide an interface device and components thereof that overcome the shortcomings of conventional interface devices and components used therein. This object is achieved according to one embodiment of the present invention by providing a customized member for use in an interface device for delivering a flow of breathing gas to the airway of a patient. The member comprises: at least one dimensional characteristic selected to fit the member to the patient; and an indicia formed permanently therein. The indicia is selected by the patient or a representative thereof.

The member may comprise a frame or a cushion.

The indicia may comprise something other than a formal name of the patient or a serial number representing the patient. The indicia may comprises a graphic. The indicia comprises a phrase. The indicia may comprise a plurality of letters. The indicia may comprise a pseudonym for the patient.

The member may comprise a first material and the indicia may comprise a second material different than the first material.

In another embodiment of the present invention an interface device for use in delivering a flow of breathing gas to the airway of a particular patient is provided. The interface device comprises: a frame, a cushion engaged with the frame, and an indicia permanently formed in at least one of the frame or the cushion. The at least one of the frame or the cushion was custom-formed for the patient during a formation process, the indicia was selected by the patient or a representative of the particular patient, and the indicia was formed during the formation process.

The indicia may comprise something other than a formal name of the patient or a serial number representing the patient. The indicia may comprise at least one of: a graphic, a phrase, a plurality of letters, or a pseudonym for the patient.

In yet another embodiment of the present invention a method of producing a customized member for use in an interface device for delivering a flow of breathing gas to the airway of a patient is provided. The method comprises: receiving an indication of a measurement of a facial dimension of the patient; receiving an indication of an indicia desired by the patient; forming the customized member in accordance with at least the measurement of the facial dimension of the patient; and forming the indicia as a portion of the customized member.

The indicia may comprise something other than a formal name of the patient or a serial number representing the patient. The indicia may comprise at least one of: a graphic, a phrase, a plurality of letters, or a pseudonym for the patient.

Forming the indicia as a portion of the customized member may comprise molding the indicia into the customized member. Forming the indicia as a portion of the customized member may comprise etching the indicia into the customized member. Forming the indicia as a portion of the customized member may comprise forming the indicia from a material which differs from a material from which the customized member is formed.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a front isometric view of a patient interface device and a portion of a conduit shown connected to a gas flow/pressure generating system (shown schematically) to form a system adapted to provide a regiment of respiratory therapy to a patient according to one exemplary embodiment of the invention;

FIGS. 2-8 are side isometric views of patient interface devices according to exemplary embodiments of the invention; and

FIG. 9 illustrates the basic steps of a method for producing a customized member for use in delivering a flow of breathing gas to the airway of a patient according to one exemplary embodiment of the invention.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

As used herein, the word “patient” or “user” shall be used interchangeably to refer to the person to which the interface device is delivering a flow of breathing gas. As used herein, the word “graphic” shall be used to refer to a graphical or pictorial representation of an object or design. As used herein, a graphic may include one or more alphanumeric characters but cannot consist solely of such characters.

As used herein, the word “pseudonym” shall be used to refer to names other than a user or patient's formal or “proper” name. For example, a user having the formal name “Thomas” may have pseudonyms such as “Tom”, “Tommy”, “T”, or any other name or nickname which the user selects.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

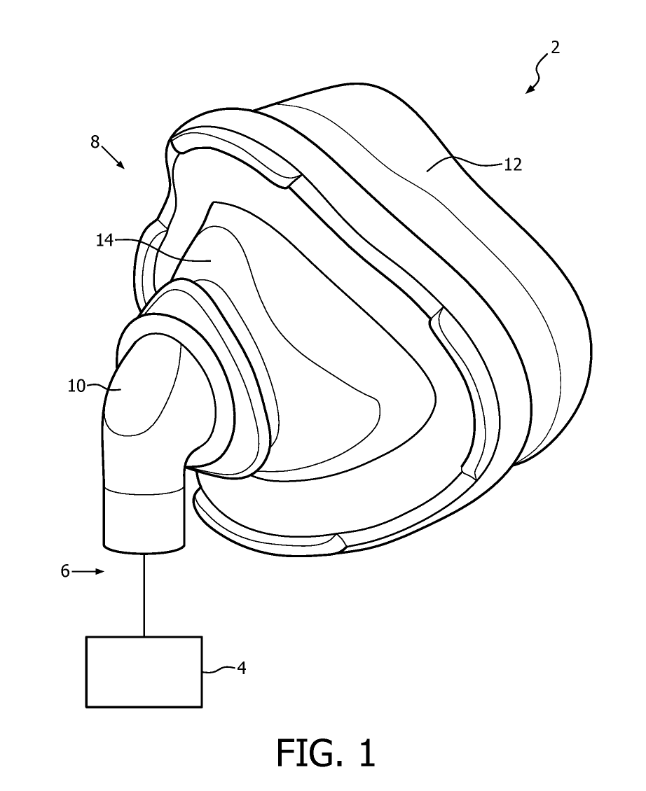

A system 2 adapted to provide a regimen of respiratory therapy to a patient according to one exemplary embodiment of the invention is generally shown in FIG. 1. System 2 includes a pressure generating device 4 (shown schematically), a delivery conduit 6 (shown partially schematically), and a patient interface device 8 having a fluid coupling conduit 10. Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 8 through fluid coupling conduit 10, which in the illustrated embodiment is an elbow connector. Delivery conduit 6 and user interface device 8 are often collectively referred to as a patient circuit.

A BiPAP® device is a bi-level device in which the pressure provided to the patient varies with the patient's respiratory cycle, so that a higher pressure is delivered during inspiration than during expiration. An auto-titration pressure support system is a system in which the pressure varies with the condition of the patient, such as whether the patient is snoring or experiencing an apnea or hypopnea. For present purposes, pressure/flow generating device 4 is also referred to as a gas flow generating device, because flow results when a pressure gradient is generated. The present invention contemplates that pressure/flow generating device 4 is any conventional system for delivering a flow of gas to an airway of a patient or for elevating a pressure of gas at an airway of the patient, including the pressure support systems summarized above and non-invasive ventilation systems.

In the exemplary embodiment illustrated in FIG. 1, patient interface device 8 is depicted as a nasal/oral mask which includes a user sealing assembly or cushion 12 coupled to a generally rigid frame 14 which is coupled to conduit 6 via fluid coupling conduit 10. However, it is to be appreciated that other types of patient interface devices, such as, without limitation, a nasal mask or a full face mask, which facilitates the delivery of the flow of breathing gas to the airway of a user, may be substituted for patient interface device 8 while remaining within the scope of the present invention. It is also to be appreciated that conduit 6 may be directly coupled to patient interface device 8 without the use of any intermediary coupling, such as conduit 10.

In exemplary embodiments of the present invention, one or both of cushion 12 or frame 14 are custom formed to fit a particular patient. As used herein, “custom formed” or “custom fitted” and variations thereof shall be used to refer to producing an element (e.g., without limitation, a cushion, frame, headgear, tubing) for a particular user such that one or more dimensional or other characteristics of the element are adapted and/or sized to provide a best fit for the particular user. For example, without limitation, one or more of the: overall dimensions, shape, size and/or shape of openings provided within, softness, hardness, and surface texture of an element may be adapted to best fit a particular user. Such custom fitment may be based on facial profile, head size, body build, personal preference(s), any combination of the aforementioned, or any other suitable basis or technique without varying from the scope of the present invention. In one example embodiment, a cushion (i.e., the sealing element) is created by matching the facial contour of the patient. In an another example embodiment, a frame which is normally more rigid than the cushion is custom formed to provide a customized support to back a commonly sized cushion to create a custom match. In yet another example embodiment, the depth of the faceplate or frame is customized to optimize the deadspace for an individual.

In order to provide an additional level of customization to a patient interface device (such as interface device 8), and thus increase the likelihood of the particular user wearing the device as proscribed, a custom indicia may be permanently formed in one or both of the cushion or frame during the formation of the element itself. In order for the indicia to be truly customized to the particular user, the indicia is selected by the user, or a representative thereof acting on behalf of the user. As used herein, a “representative” of a particular user or patient shall refer to any person or persons acting for or on behalf of the particular user. Such selection of indicia may be accomplished via any suitable means such as, for example without limitation, supplying the indicia in a reproducible form, selecting the indicia from a list or catalog of predetermined indicia, or any other suitable means. Such indicia is formed so as to be a generally permanent part of the element to which it is provided. In example embodiments according to the present invention, such indicia have been formed via molding, etching, laser-burning, ink-jetting or any other suitable means.

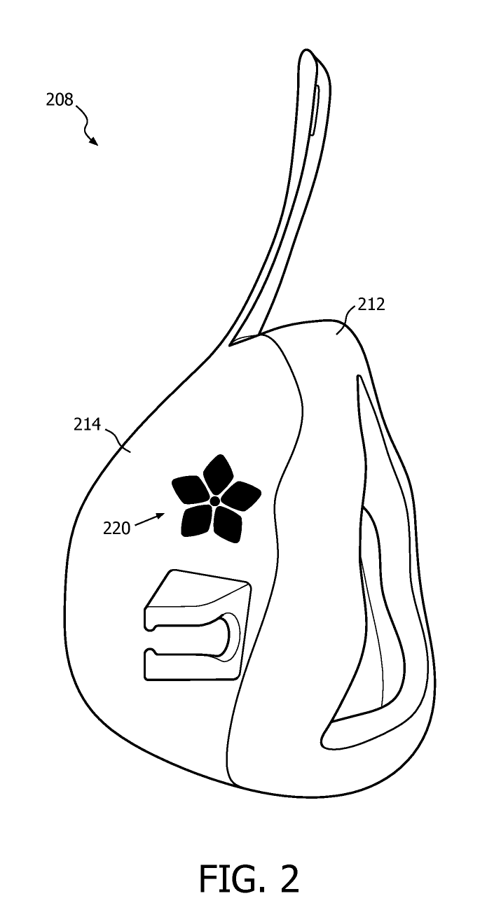

Such indicia may be formed as an indentation or raised marking from the surface of the member. The indicia could be printed in color other than the color of the majority surface of the member in the same material or in a different material which is feasible in most 3D printers. The indicia could be burnt on the surface with laser or UV beam to decolorized or otherwise darken the surface. In certain materials, a sensitive chemical can be infused into the cushion or frame materials, while a localized area of the surface is exposing to a simulant (which can be laser, UV, or chemicals) to change the surface appearance resulting in coloration marking. In the case of a fabric harness or headgear, indicia can be added by embroidery, printing, etc. It is thus to be appreciated that indicia may be formed via any suitable permanent or generally permanent processes without varying from the scope of the present invention. FIGS. 2-8 show some example embodiments of patient interface devices including indicia custom formed therein in accordance with the present invention.

FIG. 2 shows a patient interface device 208 for delivering a flow of a breathing gas to the airway of a patient in accordance with an example embodiment of the present invention. Patient interface device 208 includes a cushion 212 and a frame 214 which have been custom formed for fitting a particular patient. Frame 214 includes an indicia 220 which is in the form of a graphical or pictorial representation of a flower-like design.

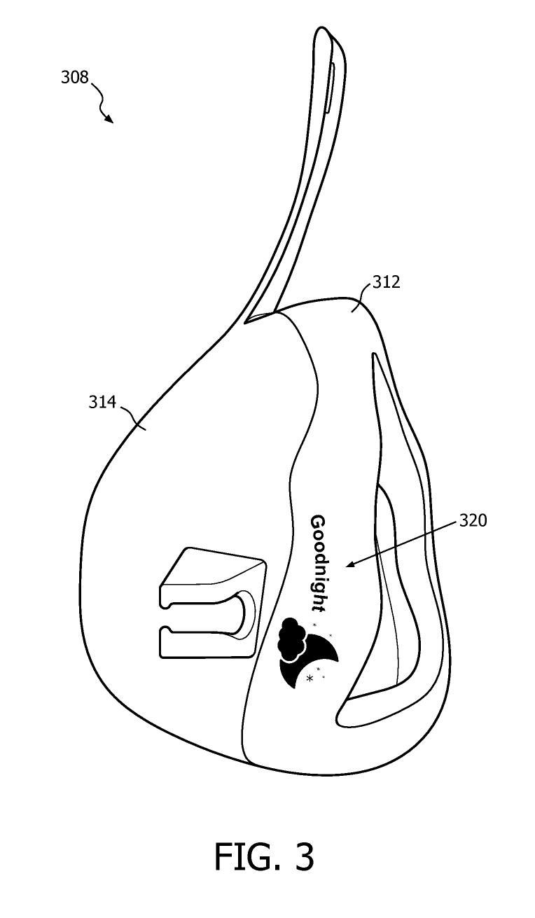

FIG. 3 shows a patient interface device 308 for delivering a flow of a breathing gas to the airway of a patient in accordance with an example embodiment of the present invention. Patient interface device 308 includes a cushion 312 which has been custom formed for fitting a particular patient and a frame 314 which can be custom formed or simply an “off-the shelf”, mass-produced element. Cushion 312 includes an indicia 320 which includes both a graphical or pictorial representations of a cloud and moon as well as the word “Goodnight”.

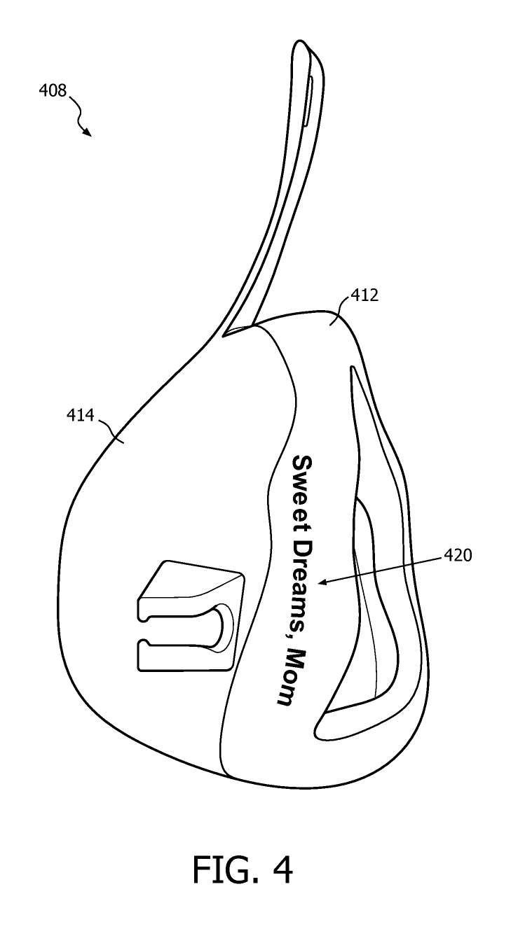

FIG. 4 shows a patient interface device 408 for delivering a flow of a breathing gas to the airway of a patient in accordance with an example embodiment of the present invention. Patient interface device 408 includes a cushion 412 which has been custom formed for fitting a particular patient and a frame 414 which can be custom formed or simply an “off-the shelf”, mass-produced element. Cushion 412 includes an indicia 420 which includes the phrase “Sweet Dreams, Mom”.

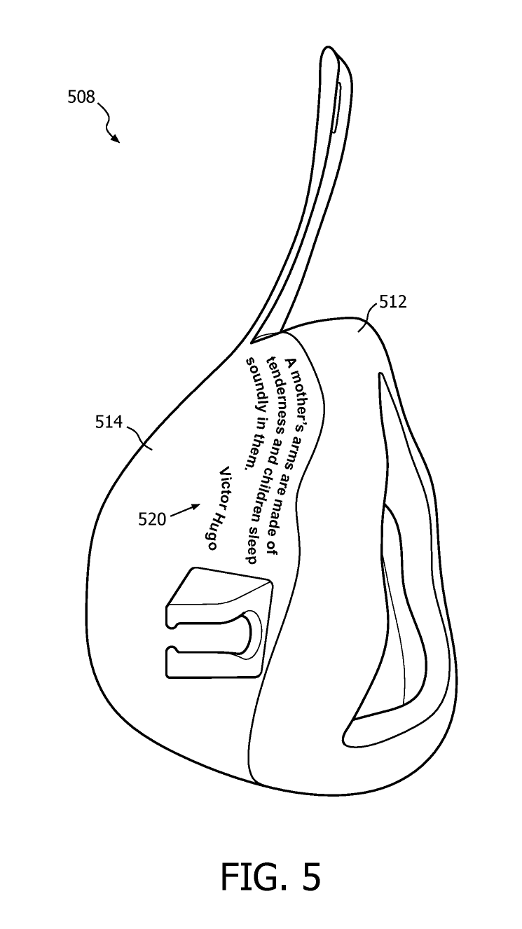

FIG. 5 shows a patient interface device 508 for delivering a flow of a breathing gas to the airway of a patient in accordance with an example embodiment of the present invention. Patient interface device 508 includes a cushion 512 and a frame 514 which have been custom formed for fitting a particular patient. Frame 514 includes an indicia 520 which is in the form of a phrase, more particularly the quote “A mother's arms are made of tenderness and children sleep soundly in them.” by Victor Hugo.

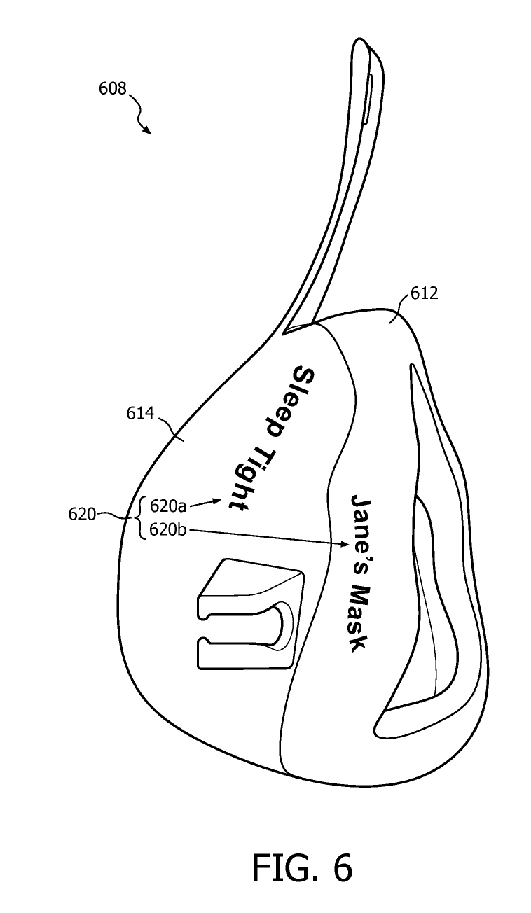

FIG. 6 shows a patient interface device 608 for delivering a flow of a breathing gas to the airway of a patient in accordance with an example embodiment of the present invention. Patient interface device 608 includes a cushion 612 and a frame 614 which have been custom formed for fitting a particular patient. Patient interface device 608 includes indicia 620 which is disposed partly on frame 614 and partly on cushion 612. Moe particularly, frame 614 includes an indicia 620a which is a phrase (i.e., “Sleep Tight”) and cushion 612 includes an indicia 620b which is a phrase (i.e., “Jane's Mask”).

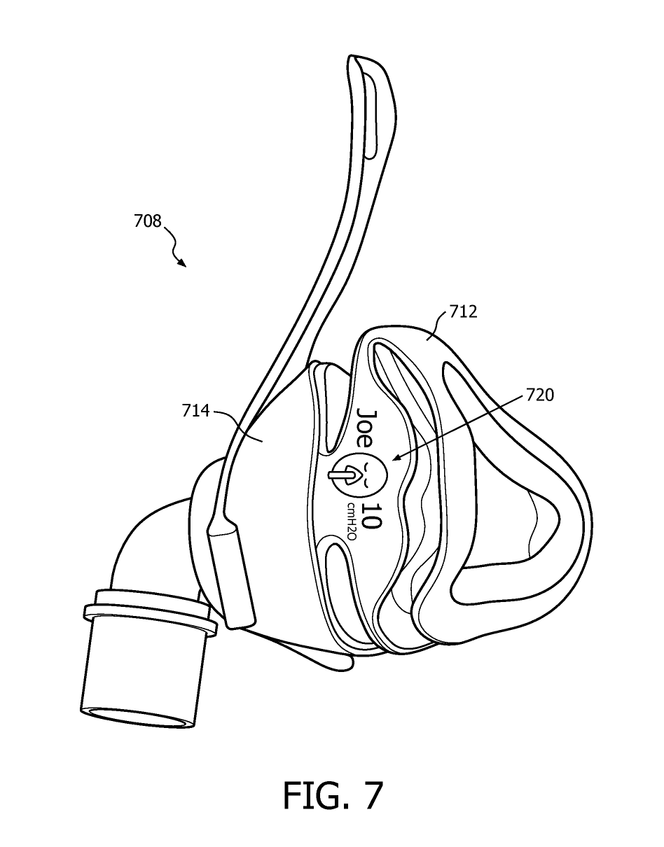

FIG. 7 shows a patient interface device 708 for delivering a flow of a breathing gas to the airway of a patient in accordance with an example embodiment of the present invention. Patient interface device 708 includes a cushion 712 which has been custom formed for fitting a particular patient and a frame 714 which can be custom formed or simply an “off-the shelf”, mass-produced element. Cushion 712 includes an indicia 720 which includes a pseudonym (i.e., “Joe”) for the particular patient, a graphic showing a cartoonish character wearing an interface device, and a proscribed deliver pressure (i.e., “10 cm H2O”).

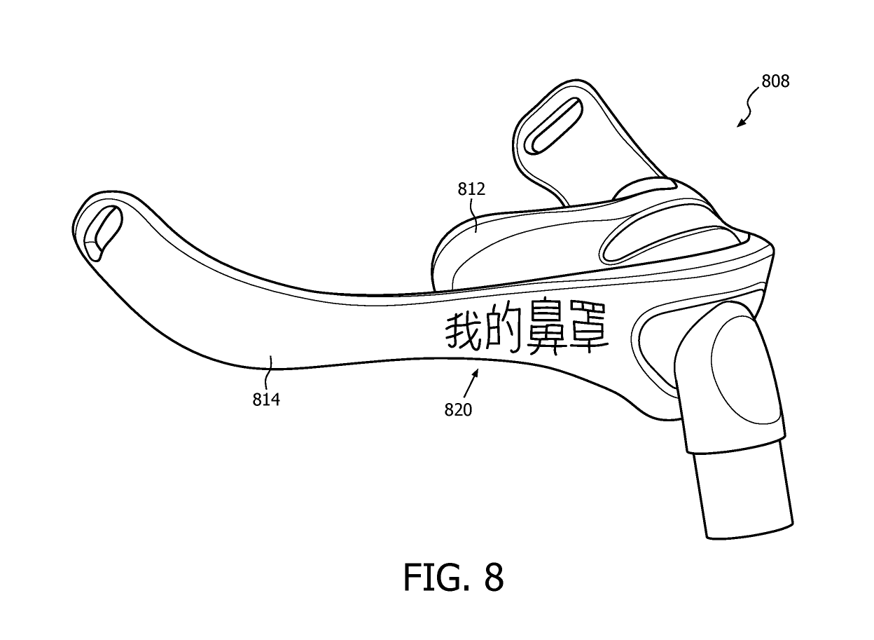

FIG. 8 shows a patient interface device 808 for delivering a flow of a breathing gas to the airway of a patient in accordance with an example embodiment of the present invention. Patient interface device 808 includes a cushion 812 and a frame 814 which have been custom formed for fitting a particular patient. Frame 814 includes an indicia 820 which is in the form of a message written in Chinese characters. It is to be appreciated, that other non-Romanized languages (e.g., without limitation, Arabic, Hebrew, Korean, and Russian) could be used without varying from the scope of the present invention.

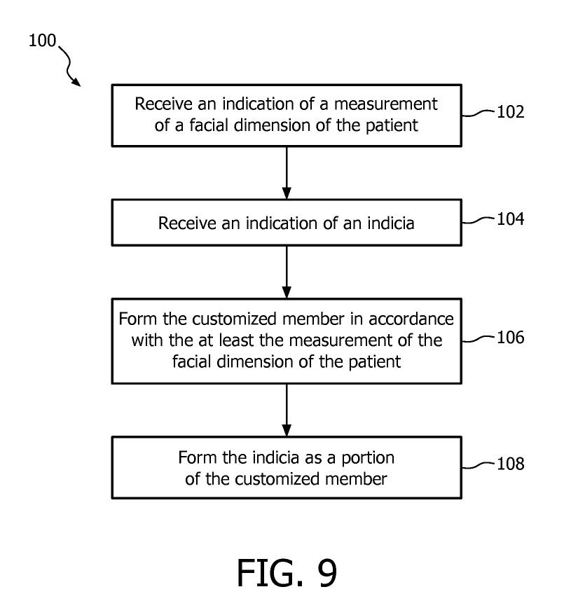

FIG. 9 illustrates the basic steps of a method 100 for producing a customized member for use in delivering a flow of breathing gas to the airway of a patient according to one exemplary embodiment of the invention. Method 100 begins at 102 by receiving an indication of a measurement of a facial dimension of the patient. Such indication may be determined/provided via any suitable means know in the art. As indicated at 104, an indication of an indicia desired by the patient is received. As previously discussed the indicia which is desired may be indicated by the patient or a representative thereof. Such indication may be made before, during, or after the facial dimension of the patient is determined/provided. As indicated at 106, the customized member is formed in accordance with at least the measurement of the facial dimension of the patient which was previously received at 102. As indicated at 108, the indicia, which was previously indicated at 108, is formed as a portion of the customized member. The indicia may be formed concurrently with member or may be formed in a subsequent operation.

From the foregoing description it is to be readily appreciated that embodiments of the present invention provide for a patient interface device which not only provides a custom fit for the particular user but also provides additional customized indicia which was personally selected by the user (or a representative thereof). By incorporating such personalized indicia, self-awareness of the of the prescribed therapy and thus compliance therewith is generally improved. It is also to be appreciated that indicia formed in accordance with the present invention may be provided as a portion of one or more elements in a single interface device and that such indicia as described herein is of a personal nature and thus constitutes more than merely a formal name of the patient or a serial number representing the patient.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A customized member for use in an interface device for delivering a flow of breathing gas to the airway of a patient, the member comprising:
at least one dimensional characteristic selected to fit the member to the patient; and
an indicia formed permanently therein, the indicia being selected by the patient or a representative thereof.

2. The member of claim 1, wherein the member comprises a frame or a cushion.

3. The member of claim 1, wherein the indicia comprises something other than a formal name of the patient or a serial number representing the patient.

4. The member of claim 1, wherein the indicia comprises a graphic.

5. The member of claim 1, wherein the indicia comprises a phrase.

6. The member of claim 1, wherein the indicia comprises a plurality of letters.

7. (canceled)

8. The member of claim 1, wherein the member comprises a first material and the indicia comprises a second material different than the first material.

9. An interface device for use in delivering a flow of breathing gas to the airway of a particular patient, the interface device comprising:
a frame;
a cushion engaged with the frame; and
an indicia permanently formed in at least one of the frame or the cushion, wherein the at least one of the frame or the cushion has been custom-formed for the patient during a formation process, wherein the indicia was selected by the patient or a representative of the particular patient, and wherein the indicia was formed during the formation process.

10. (canceled)

11. The interface device of claim 9, wherein the indicia comprises at least one of: a graphic, a phrase, a plurality of letters, a pseudonym for the patient, or something other than a formal name of the patient or a serial number representing the patient.

12. A method of producing a customized member for use in an interface device for delivering a flow of breathing gas to the airway of a patient, the method comprising:
receiving an indication of a measurement of a facial dimension of the patient;
receiving an indication of an indicia desired by the patient;
forming the customized member in accordance with at least the measurement of the facial dimension of the patient; and
forming the indicia as a portion of the customized member.

13. The method of claim 12, wherein the indicia comprises something other than a formal name of the patient or a serial number representing the patient.

14. The method of claim 12, wherein the indicia comprises at least one of: a graphic, a phrase, a plurality of letters, or a pseudonym for the patient.

15. The method of claim 12, wherein forming the indicia as a portion of the customized member comprises molding the indicia into the customized member.

16. The method of claim 12, wherein forming the indicia as a portion of the customized member comprises etching the indicia into the customized member.

17. The method of claim 12, wherein forming the indicia as a portion of the customized member comprises forming the indicia from a material which differs from a material from which the customized member is formed.
